SEQUENCE LISTING (5 sequences)

SEQ ID NO: 1 - protein, 23 aa, Homo sapiens
APPTYDTVLQMEYLDMVVNETLR

SEQ ID NO: 2 - protein, 21 aa, Homo sapiens
VWGFYDGQQPVLAITDPDMIK

SEQ ID NO: 3 - protein, 7 aa, Homo sapiens
EVTNFLR

SEQ ID NO: 4 - protein, 10 aa, Homo sapiens
RPFGPVGFMK

SEQ ID NO: 5 - protein, 16 aa, Homo sapiens
LSLGGLLQPEKPVVLK

(19) United States
(12) Patent Application Publication
(10) Pub. No.: US 2021/0372996 A1
(43) Pub. Date: Dec. 2, 2021

(54) METHODS FOR ASSESSING EFFICACY OF A DRUG IN VIVO BY MONITORING ADME PROTEINS IN EXTRACELLULAR VESICLES

(71) Applicant: Purdue Research Foundation, West Lafayette, IN (US)

(72) Inventors: Weiguo Tao, West Lafayette, IN (US); Xiaofeng Wu, West Lafayette, IN (US)

(21) Appl. No.: 17/335,578

(22) Filed: Jun. 1, 2021

Related U.S. Application Data
(60) Provisional application No. 63/032,786, filed on Jun. 1, 2020.

(51) Int. Cl. G01N 33/50; G01N 33/68; C12Q 1/26

(52) U.S. Cl. CPC G01N 33/5088; G01N 33/6842; C12Q 1/26; C12Y 114/13032; G01N 2570/00; G01N 2333/90251; G01N 2333/46

(57) ABSTRACT

Materials and non-invasive methods are provided for determining a subject's present drug metabolic capacity by assessing a biofluid sample taken from the subject following administration of a compound and identifying phenotypic variations in activity of one or more drug absorption, distribution, metabolism and excretion (ADME) enzymes extracted from extracellular vesicles (EVs) in the biofluid sample. Additional materials and methods are provided for determining compound (e.g., drug) efficacy and dose ranging, either in a subject or in a treatment cohort, by quantifying level of expression of such ADME enzymes EVs selectively isolated from biofluid.

### PRIORITY

This application is related to and claims priority benefit of U.S. Provisional Patent Application Ser. No. 63/032,786 to Tao et al. filed Jun. 1, 2020. The contents of the aforementioned application is hereby incorporated by reference in its entirety into this disclosure.

### FIELD

This disclosure relates to materials and methods for determining an individual subject's particular drug response and effective dosage requirements by identifying phenotypic variations in each subject's metabolic capabilities through the assessment of drug absorption, distribution, metabolism and excretion enzymes extracted from biofluid-derived extracellular vesicles.

### p BACKGROUND

The primary goal of drug development is to obtain a compound that has a therapeutic effect in a form that can be used to safely and effectively dose patients. Potential drugs require appropriate pharmacokinetic properties to be safe, useable, and effective therapeutics. Accordingly, a drug must be able to be absorbed into a subject's bloodstream, reach the site of action, remain unchanged long enough to exert a pharmacological/therapeutic effect and then be converted into safe metabolites, and be cleared from the subject within a reasonable timeframe. Characterization of drug absorption, distribution, metabolism and excretion (ADME) properties is commonly used to evaluate these criteria through the assessment of the pharmacokinetics and pharmacological activity of a compound both in vitro and in vivo.

Absorption is the process by which a drug moves from the administration site into the bloodstream. While there are multiple well-known routes of administration, only injected compounds enter directly into systemic circulation. For drugs administrated through ingestion, inhalation or dermal contact, for example, the compounds must cross a membrane before entering the bloodstream. For example, small molecules may traverse membranes via passive transport or by way of proteins known as drug transporters. A drug's absorption and, thus, bioavailability can be significantly influenced by many factors including, without limitation, route of administration, molecular weight, topological polar surface area, solubility, ionization, and other physiochemical properties of the compound. m

When a drug has been absorbed, it moves from the absorption site to tissues around the body. This distribution from one part of the body to another is typically accomplished via the bloodstream, but it can also occur from cell-to-cell transfer. Drug developers can obtain a big-picture view of drug concentration in various tissues and organs over time from radiolabeled in vivo ADME studies, which conventionally include quantitative whole-body autoradiography, microautoradiography, and tissue dissection. In vitro studies can also be used to obtain more minute details of a compound's distribution. Factors such as blood flow, lipophilicity, tissue binding, and molecular size can influence drug distribution.

Drug metabolism is typically responsible for converting drugs to compounds that are more water soluble and more easily excreted but may also be involved in the conversion of prodrugs into active compounds or drugs to toxic metabolites. Because metabolism can result in toxicity, for instance by formation of toxic or biologically reactive metabolites, scientists typically map out the specific metabolic pathways of a drug candidate (i.e. adverse outcome pathways) to provide data on the potential safety or toxicity of a drug. Drug metabolism and interaction data can provide information to validate major players in a drug's metabolism.

A drug's excretion path and rate are also important ADME factors. Excretion of drugs (including parent drug and metabolites) commonly occurs by function of the kidney (urine) or liver (bile/feces), but drugs can also be excreted through sweat, tears, or breath. Molecular size and charge can influence the excretion pathway.

The pharmacokinetics of a drug describe a drug's exposure through characterization of ADME properties as a function of time. With the exception of drugs delivered intravenously, only a fraction of a drug's dose is absorbed and pharmacologically active. Quantifying the rate and magnitude of exposure to a drug is critical for determining how to best guide its use in clinic; however, not all patients respond the same with identical drug exposure. Mammals, and humans in particular, are highly variable in their ability to metabolize and excrete drugs.

Indeed, the drug concentration in a patient's blood/serum is dependent on both drug dosage and the patient's metabolic behavior. Genetic variation on drug metabolizing enzymes between patients can significantly affect a patient's response to drugs including, for example, drug metabolism. For example, an average dosage can be possibly toxic to a poor metabolizer because that patient is not efficient in detoxifying and excreting drugs. While drug dosage has conventionally been determined by research results and clinical studies, methods for noninvasively assessing patient-specific metabolic behavior (e.g., poor/intermediate/extensive/ultra-rapid) have not been, to date, readily available.

Accordingly, there is a need for improved methods for accurately identifying and predicting drug toxicity and response on a patient-specific basis to help guide drug testing, clinical treatment plans and/or drug dosing regimens. Such methods should ideally be easy and cost effective to perform in a clinical setting, noninvasive, accurate, and provide rapid results.

### BRIEF SUMMARY

Methods are provided for determining efficacy or a dosing range of a compound (e.g., a pharmaceutical compound or drug). In at least one embodiment, such a method comprises selectively isolating a population of extracellular vehicles (EVs) in a biofluid sample of a subject having received at least one dose of a compound; extracting EV proteins from the isolated population of EVs; enriching the extracted EV proteins or peptides generated from the extracted EV proteins; and identifying and quantifying a level of expression of one or more targeted absorption, distribution, metabolism, and excretion (ADME) proteins in the enriched EV proteins or peptides. In at least one embodiment, the enriching step comprises enriching the extracted EV proteins or peptides generated from the extracted EV proteins using affinity purification. In at least one embodiment, the affinity purification comprises immobilized metal affinity chromatography or immunoaffinity chromatography. Still further, the extracting step can be performed via lipid affinity-based capture (e.g., using an EVTRAP device). In certain embodiments, the identifying and quantifying step is performed by multi-reaction monitoring, parallel reaction monitoring, or data-independent acquisition mass spectrometry.

The upregulated expression of the targeted one or more ADME proteins in the subject as compared to a baseline expression of the targeted one or more ADME proteins is indicative of metabolism by the subject of the compound and the at least one dose of the compound being within the therapeutic index of the compound for the subject. Additionally, the baseline expression of the targeted one or more ADME proteins in the subject can be indicative of the at least one dose of the compound being outside of the therapeutic index of the compound for the subject.

The method can further comprise adjusting a dosing range of the compound of the targeted one or more ADME proteins if the targeted one or more ADME proteins in the subject equates with baseline expression. Certain embodiments of the method also comprise administering an adjusted dose of the compound to the subject if the targeted one or more ADME proteins has baseline expression in the subject.

The step of extracting EV proteins can further comprise generating EV peptides therefrom. In at least one embodiment of the method, the one or more targeted ADME proteins can each have a sequence comprising one of SEQ ID NOS. 1-5.

The biofluid sample can be selected from a group consisting of urine, blood, cerebrospinal fluid, plasma, serum, tears, saliva, sweat, amniotic fluid, breast milk, ascites fluid, bile and cyst fluid. In at least one exemplary embodiment, the biofluid sample comprises blood.

The subject can be an individual in a treatment cohort. In other embodiments, the subject can be a patient in, for example, a clinical setting.

In certain embodiments, the method can further comprise identifying the subject's phenotype for drug metabolism based on the quantified level of expression of the one or more targeted ADME proteins.

Methods are also provided for profiling a subject's present drug response phenotype. In at least one embodiment, such a method for profiling a subject's present drug response phenotype comprises: selectively isolating a population of EVs in a biofluid sample of a subject that has received at least one dose of a first compound; extracting EV proteins from the isolated population of EVs; enriching the extracted EV proteins or peptides generated from the extracted EV proteins; and identifying and quantifying a level of expression, using parallel reaction monitoring, of one or more targeted drug ADME proteins in the enriched EV proteins or peptides; and assigning the subject a phenotype for drug metabolism of poor, intermediate, extensive or ultra-rapid based on the level of expression of the targeted one or more ADME proteins. Additionally, the method can further comprise administering an adjusted dose of the first compound or administering at least one dose of a second compound based on the assigned phenotype.

In at least one embodiment, the biofluid sample is blood, the step of enriching comprises enriching the extracted EV proteins or peptides using affinity purification, and the step of extracting is performed via lipid affinity-based capture.

The step of extracting can be performed using a surfactant-based solution, a triethylamine elution, on-beads digestion, a high temperature elution, or electronic pulse-based extraction.

The targeted one or more ADME proteins can comprise enzymes from the cytochrome P450 superfamily of hemoproteins (CYP), enzymes from the UDP-glucuronosyltransferse (UGT) family, or both CYP and UGT enzymes. In at least one embodiment, the targeted one or more ADME proteins each have a sequence comprising one of SEQ ID NOS: 1-5. In certain embodiments, the targeted one or more ADME proteins comprise CYP enzymes, UGT enzymes, acetyltransferase, an ATP-binding cassette transporter, and a solute carrier transporter.

Methods for determining drug efficacy are also provided, such methods comprising: isolating a population of EVs in a biofluid sample of a subject having received at least one dose of a compound; extracting EV proteins from the isolated population of EVs; enriching the extracted EV proteins or peptides generated from the extracted EV proteins; and identifying and quantifying a level of expression of one or more targeted drug ADME proteins in the enriched EV proteins or peptides, where the upregulated expression of the targeted one or more ADME proteins in the subject as compared to a baseline expression of the targeted one or more ADME proteins is indicative of the at least one dose of the compound being within the therapeutic index of the compound for the subject. Additionally, the baseline expression of the targeted one or more ADME proteins in the subject can be indicative of the at least one dose of the compound being outside of the therapeutic index for the subject.

In at least one exemplary embodiment, the isolating step of the method for determining drug efficacy is performed via lipid affinity-based capture using a device comprising a plurality of microspheres having surfaces coated or modified with a combination of at least one hydrophilic group and at least one hydrophobic group (e.g., an EVTRAP). Additionally, the step of identifying and quantifying can be performed by multi-reaction monitoring, parallel reaction monitoring, or data-independent acquisition mass spectrometry.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1A shows a chart descriptive of the effects of free drug concentration in circulating blood/serum of a subject;

FIG. 1B is a representation of a eukaryotic cell and the synthesis of extracellular vesicles (EVs) therefrom;

FIG. 2 shows an illustrative representation of a differential ultracentrifugation technique for extracellular vesicle (EV) isolation from a biofluid;

FIG. 3 is a chart illustrating the advantages of the EVTRAP isolation method as compared to traditional differential ultracentrifugation (DUC) techniques;

FIG. 4 shows comparative data related to EVTRAP performance in isolating EVs from a sample as compared to isolation via DUC, wherein the EVs were shed from a PANC-1 cell line (human pancreas/duct cells) and the sample comprised cell culture media with 10% EV-free fetal bovine serum in Dulbecco's Modified Eagle Medium (DMEM);

FIG. 5 shows comparative data related to EVTRAP and DUC performance in isolating mouse liver tissue-derived EVs, with abundances from 100K normalized to 1;

FIGS. 6A-6D show data related to EV total recovery and purification from human plasma samples using the EVTRAP, with FIG. 6A showing comparative data related to EVTRAP and DUC performance in isolating EVs in 5 μl samples of human plasma, FIG. 6B showing comparative data related to recovery of various extraction methodologies (shown using CD9 signal comparisons, with n=3), FIG. 6C showing a gel, with silver staining underlying the high purity of the EVTRAP, and FIG. 6D summarizing the elevated EV protein abundance obtained from the EVTRAP technique with contaminant-free proteins remaining at the same level;

FIG. 7A show bar graphs that depict the PRM quantitative results on hepatocyte lysate ADME drug induction, using CYP3A4 (perhaps more specifically, unique CYP3A4 peptide SEQ ID NO: 3) as a protype of ADME;

FIG. 7B shows a bar graph that depicts the same experiment as FIG. 7A, however assessed using DDA mode;

FIGS. 8A and 8B are graphs representative of PRM quantitative results on plasma EV ADME (SEQ. ID NO: 4 in FIG. 8A and SEQ ID NO: 5 in FIG. 8B), using CYP3A4 as a prototype ADME;

FIG. 9 is a workflow of a method comprising EV CYP3A4 enrichment by immobilized metal ion affinity capture (IMAC);

FIG. 10 shows an SDS-PAGE gel illustrating the results of the enrichment portion of the workflow of FIG. 9 performed on biological samples, the samples prepared as follows: 25 μg=5 μg recombinant CYP3A4+20 μg K562 cell lysate (20% CYP3A4 in total mixture), and with the loading buffer being 50 mM Tris-HCL, 300 mM NaCL, pH-8, the washing buffer #1 being the same as the loading buffer, the washing buffer #2 being 300 mM imidazole, the washing buffer 43 being 100 mM ethylenediamirietetraacetic acid (EDTA). and the elution boiling LDS buffer;

FIG. 11 is a workflow of a method comprising EV CYP3A4 enrichment by antibodies (immunoprecipitation (IP)); and

FIG. 12 shows an SDS-PAGE gel illustrating the results of the enrichment portion of the workflow of FIG. 11 performed on biological samples.

While the present disclosure is susceptible to various modifications and alternative forms, exemplary embodiments thereof are shown by way of example in the drawings and are herein described in detail.

### BRIEF DESCRIPTION OF THE SEQUENCE LISTINGS

SEQ ID NO: 1 is an amino acid sequence for a first CYP3A unique peptide biomarker: APPTYDTVLQMEYLDMVVNETLR;

SEQ ID NO: 2 is an amino acid sequence for a second CYP3A unique peptide biomarker: VWGFYDGQQPVLAITDPDMIK;

SEQ ID NO: 3 is an amino acid sequence for a third CYP3A unique peptide biomarker: EVTNFLR;

SEQ ID NO: 4 is an amino acid sequence for a fourth CYP3A unique peptide biomarker: RPFGPVGFMK; and

SEQ ID NO: 5 is an amino acid sequence for a fifth CYP3A unique peptide biomarker: LSLGGLLQPEKPVVLK.

The above-described sequences are set forth in the Sequence Listing Section below and also provided in computer readable form encoded in a file filed herewith and herein incorporated by reference. The information recorded in computer readable form is identical to the written Sequence Listings provided herein, pursuant to 37 C.F.R. § 1.821(f).

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, systems and methods hereof may comprise many different configurations, forms, materials, and accessories.

In the following description, numerous specific details are set forth to provide a thorough understanding of the present disclosure. Particular examples may be implemented without some or all of these specific details and it is to be understood that this disclosure is not limited to particular biological systems, which can, of course, vary.

As used herein, the terms “detecting,” “detected,” and “detection” each refer to confirming the presence of a detectable moiety by observing the occurrence of a detectable signal, such as a radiologic, colorimetric, fluoroscopic, chemiluminescent, or spectroscopic signal that will appear exclusively in the presence of the detectable moiety.

As used herein, a “subject” is a mammal, preferably a human. A subject can also be a non-human animal (including, without limitation, a laboratory, an agricultural, a domestic, or a wild animal). Thus, the methods described herein are applicable to both human and veterinary applications. In various aspects, the subject can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, or a goat, or a wild animal in captivity such as a bear, a panda, a lion, a tiger, a leopard, an elephant, a zebra, a giraffe, a gorilla, a dolphin, or a whale. In certain embodiments, subjects are “patients,” i.e., living humans or animals that are receiving medical care for a disease or condition, which includes persons or animals with no defined illness who are being evaluated for signs of pathology or prophylactic treatment. In certain embodiments, subjects can include individuals that are part of a cohort being evaluated for drug safety and/or efficacy studies.

“Biofluid” means any bio-organic fluid produced by a subject including, without limitation, blood, plasma, serum, urine, tears, sweat, saliva, cerebrospinal fluid (C SF), amniotic fluid, breast milk, ascites fluid, bile, and cyst fluid.

The terms “treatment” or “therapy” include curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of the following: halting the onset, reducing the risk of development, reducing the incidence, delaying the onset, reducing the development, and increasing the time to onset of symptoms of a particular disorder.

As used herein, a “drug” is a compound (e.g., a medicine) or other substance that can have physiological effect when ingested or otherwise introduced into a subject.

The term “therapeutically effective dose” means (unless specifically stated otherwise) a quantity of a compound/drug that, when administered either one time or over the course of a treatment cycle affects the health, wellbeing, or mortality of a subject (e.g., and without limitation, delays the onset of and/or reduces the severity of one or more of the symptoms associated with an active infection or cervical cancer). The amount of the compound to be administered to a recipient will depend on the type of disease being treated, how advanced the disease pathology is, and the characteristics of the subject (such as general health, age, sex, body weight, and tolerance to drugs).

A “cohort” is a group of individuals identified by a set of one or more characteristics. This group can be studied over a period of time as part of a scientific study, for example, a drug efficacy, drug safety, and/or drug dosage range study. A “treatment cohort” is a cohort selected for a particular action or treatment (e.g., receiving a dosage or range of doses of a compound to study its therapeutic effects or dosage range). A “control cohort” is a group selected from a population that is used as a control. The control cohort is observed under ordinary conditions while another group (i.e. the treatment cohort) is subjected to the treatment or other factor being studied. The data from the control cohort can be the baseline against which experimental results can be measured.

“Down-regulation” or “down-regulated” may be used interchangeably and refer to a decrease in the level of a marker, such as a gene, nucleic acid, metabolite, transcript, protein, or polypeptide, as compared to an established level (e.g., that of a healthy cohort or the subject of interest). “Up-regulation” or “up-regulated” may also be used interchangeably and refer to an increase in the level of a marker, such as a gene, nucleic acid, metabolite, transcript, protein, or polypeptide, as compared to an established level (e.g., that of a healthy control or the subject of interest).

A “marker” or “biomarker” as the terms are used herein may be described as being differentially expressed when the level of expression in a subject who is experiencing an active disease state is significantly different from that of a subject or sample taken from a healthy subject. A differentially expressed marker may be overexpressed or underexpressed as compared to the expression level of a normal or control sample or subjects' baseline (i.e. down-regulated). The increase or decrease, or quantification of the markers in a biological sample may be determined by any of the several methods known in the art for measuring the presence and/or relative abundance of a gene product or transcript. The level of markers may be determined as an absolute value, or relative to a baseline value, and the level of the subject's markers compared to a cutoff index. Alternatively, the relative abundance of the marker or markers may be determined relative to a control, which may be a clinically normal subject.

A “profile” or “assay” or “panel” is a set of one or more markers and their presence, absence, and/or relative level or abundance (relative to one or more controls). For example, a panel of ADME proteins is a dataset of the presence, absence, relative level or abundance of the target proteins of interest present within a sample. A genomic or nucleic acid profile is a dataset of the presence, absence, relative level or abundance of expressed nucleic acids (e.g., transcripts, mRNA, or the like). A profile may alternatively be referred to as an expression profile or expression pattern.

As used herein, the term “point of care” or “POC” means the point in time when clinicians or other healthcare providers delivery healthcare products and services to patients at the time of care. Diagnostic testing that occurs at POC is performed at or near the point of care/bedside (as compared to historical testing which was wholly or mostly confined to the medical laboratory—i.e. sending specimens away).

The terms “isolate” and “isolated” means that the material is removed from its original environment, e.g., the natural environment if it is naturally occurring. For example, a naturally-occurring polypeptide present within a living organism is not isolated, but the same polypeptide separated from some or all of the coexisting materials in the natural system is isolated.

The term “purified” does not require absolute purity; instead, it is intended as a relative definition.

The present disclosure provides materials and non-invasive methods for determining an individual subject's current drug response by identifying phenotypic variations in such subject's metabolic capabilities of pharmaceutical compounds, drugs, or other compounds. Additional materials and methods are also provided for determining compound (e.g., drug) efficacy and dose ranging. These methods comprise minimally invasive “liquid biopsies” and, in other words, assess drug absorption, distribution, metabolism and excretion (ADME) enzymes extracted from biofluid-derived extracellular vesicles (EVs). The methods hereof are non-invasive, rapid, and conducive to being performed in a clinical setting and, as such, can be utilized at point of care (e.g., in routine clinical applications to precisely tune drug dosage to a specific subject/patient) or in pharmaceutical efficacy studies (e.g., to quickly determine dose-response relationships).

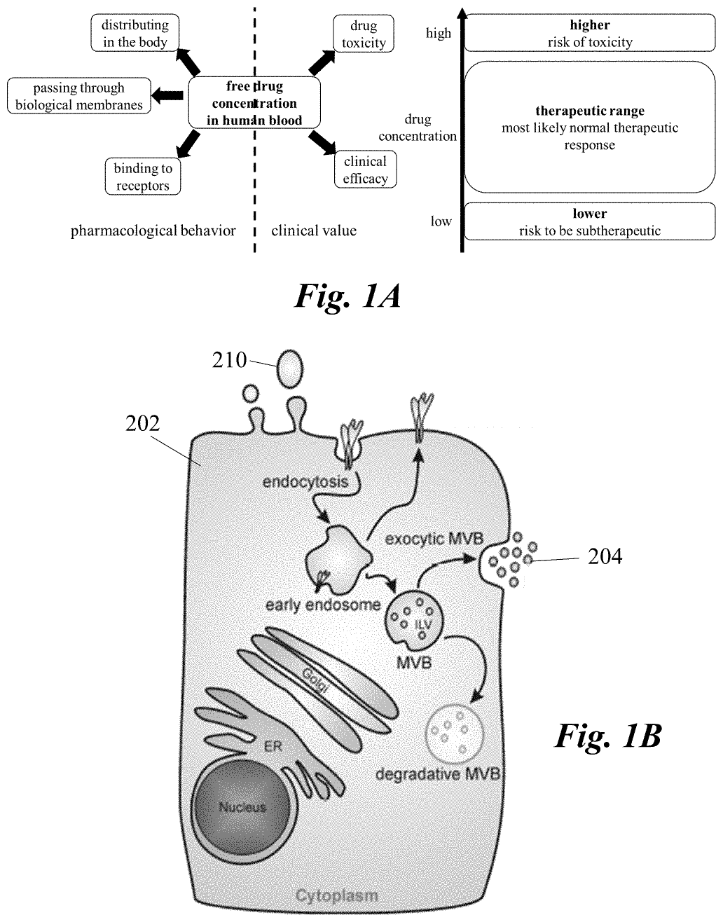

Regardless of how a drug effect occurs—through binding or chemical interactions—the concentration of the drug at the site of action (often the subject's blood/serum) controls the effect. Dose-response determines the required dose and frequency as well as the therapeutic index of a drug in a population. The therapeutic index (TI) (or therapeutic ratio or range)—the ratio of the minimum toxic concentration to the median effective concentration—helps determine the efficacy and safety of a drug. The TI provides a therapeutic window that includes a range of doses which optimize between efficacy and toxicity, achieving the greatest therapeutic benefit without resulting in unacceptable side-effects or toxicity. As illustrated in FIG. 1A, within the TI range, the drug tends to be efficient; however, increasing the dose of the drug above the range increases the probability of toxicity and decreasing the dose of the drug below the range increases the probability that the drug will be therapeutically ineffective.

However, response to concentration can be complex and is often nonlinear due, at least in part, to subject-specific factors such as a subject's drug metabolic capabilities. Because mammals (humans in particular) are highly variable in their ability to metabolize and excrete drug, the relationship between the drug dose, regardless of administration route used, and the drug concentration at the cellular level is complex.

A subject's degree of drug metabolism can be significantly influenced by the activity of ADME protein functions, with at least the cytochrome P450 superfamily of hemoproteins (CYP) and UDP-glucuronosyltransferase (UGT) families of drug metabolizing enzymes playing essential roles. Assessing these ADME-associated proteins can provide the opportunity to account for subject variabilities in drug exposure. Among them, the CYP and UGT enzymes together account for the metabolic clearance of greater than 90% of drugs that are subject to metabolism. Accordingly, the activity of CYP and UGT enzymes is an important determinant of drug exposure in the body.

The activity of CYP and UGT enzymes is determined by their intrinsic function and expression. Conventional attempts to explain variability in ADME enzyme activities have largely focused on the assessment of genotype differences via a pharmacogenomics approach; however, genotype alone is insufficient to accurately predict subject exposure to a drug as it cannot cover all the possible alleles/polymorphisms of enzyme gene codes, nor can the phenotype be predicted solely according to genes.

For example, CYP3A4 is a well-known drug-metabolizing enzyme of clinical importance as it is involved in the metabolism of more than 30% of all drugs, including immunosuppressants, chemotherapeutics, macrolide antibiotics, antidepressants, anxiolytics, antipsychotics, opiates, calcium channel blockers, and statins. However, wide variability in CYP3A4 activity is largely driven by differences in protein expression (not genetic polymorphisms), which is poorly described by conventional genotype- and biomarker-based approaches. Another example is CYP2A6, a primary enzyme responsible for the oxidation of nicotine and cotinine. The enzyme is also involved in the metabolism of several pharmaceuticals, carcinogens, and a number of coumarin-type alkaloids.

While assessment of hepatic CYP expression (or their mRNA expression) has potential as a robust biomarker related to variability in CYP3A activity, conventional assessments have significant drawbacks. Indeed, surgical resected liver tissue is required to determine expression of hepatic CYP3A mRNAs using conventional techniques, which is preclusively invasive. Attempts to correlate CYP3A4 mRNA expression in more diagnostically amenable samples (e.g., peripheral blood mononuclear cells (PBMCs)) with activity or induction have demonstrated that PBMC mRNA expression does not correlate with CYP3A4-mediated drug clearance.

Additionally, grapefruit juice, for example, can be a powerful inhibitor to CYP3A4. Consider a subject that, from a genotypic perspective, presents as a normal drug metabolizer, but intakes a drug meant to be metabolized by CYP3A4 after ingesting grapefruit juice. Because the grapefruit juice can inhibit the CYP3A4, such subject could be at significant risk for drug toxicity because the effect of the inhibitor (grapefruit juice) has at least temporarily modified the subject's CYP3A4 phenotype.

In recent years, EV-derived biomarkers have revolutionized the diagnosis of multiple pathologies including cancer, cardiovascular disease and liver injury. EVs are important biological carriers for intercellular communications and are produced by cells found in all domains of life including complex eukaryotes, both Gram-negative and Gram-positive bacteria, mycobacteria, and fungi. Generally, and as illustrated in FIG. 1B, EVs 204 are membrane-encapsulated nano- or microparticles that are naturally released from a cell 202 and, unlike a cell, cannot replicate. In mammals, EVs are shed by cells of all types and can be found in all biofluids and sometimes in intercellular regions.

The major roles of EVs are to transfer information from the original cell to other cells using different classes of molecules (i.e. intercellular communication) and to remove excess materials. EVs 204 carry a cargo of proteins, nucleic acids, lipids, metabolites, and even organelles from the parent cell 202, with such cargo being protected from external proteases, phosphatases, and other enzymes by the outer membrane of the EV. Accordingly, EVs are highly stable in biofluid for extended periods of time.

There are many types of EVs 204 ranging in diameter from around 20-30 nm to as large as 10 microns or more, although the vast majority of EVs 204 have a diameter of less than about 200 nm. A wide variety of EV subtypes have been proposed, defined variously by size, biogenesis pathway, cargo, cellular source, and function. For example, exosomes are membrane bound extracellular vesicles of endocytic origin (about 30 nm-150 nm in diameter), which are formed by complex endocytosis, encapsulation of compounds and secretion by cells. Exosomes (including exosome-like vesicles) have been isolated and characterized from different biological fluids such as urine, BAL fluid, and serum. Microvesicles 210 (also referred to as shedding microvesicles (SMVs)) are shed directly from the plasma membrane and are typically between about 100 nm-1 μM in diameter. Membrane particles (about 50-80 nm in diameter), or large membranous vesicles (about 600 nm in diameter) can include CD133⁺ and CD63⁺. Apoptotic blebs/bodies or vesicles (not shown) (about 1000-5000 nm in diameter) are released by dying cells undergoing apoptosis. Since apoptotic cells tend to display phosphatidylserine (PS) in the outer bilayer of the cell membrane, apoptotic bodies tend to externalize PS and tend to be quite large (e.g., microns in diameter).

EVs' 204 cargo (including, for example, proteins, phosphoproteins, nucleic acids (e.g., double stranded DNA, mRNA, and miRNA), lipids, metabolites, and even organelles from the parent cell) is information-rich and reflects the functional state of the parent cell 202. Analysis of the EV cargo has a great potential for biomarker discovery and disease diagnosis. For example, considering EVs in the context of tumor biology and cancer, there is strong evidence that EV-based disease markers can be identified well before the onset of symptoms or physiological detection of a tumor, making EVs 204 prime candidates for early cancer detection biomarkers ahead of conventional nuclear imaging studies. Additionally, EVs have been found to circulate through many different biofluids, including blood and urine as previously noted. Due to the resemblance of EVs composition with the parental/host cell and because collecting biofluids in many instances is non-invasive, circulating EVs have raised considerable interest as a source for the discovery of biomarkers.

Since EVs are membrane covered nanoparticles and their content is protected from external proteases, enzymes and phosphatases, they are promising candidates for biomarker discovery. Additionally, due to large dynamic ranges and the presence of phosphatases and enzymes in the blood, EV content is very valuable for diagnostics/prognostics of diseases like cancer. However, very limited data has been conventionally available on sequential enrichment of the cargo in EVs, at least in part because of the limited amounts of purified EVs, low-abundance/trace amounts of proteins that can be extracted, and interference from proteins and metabolites in biofluids. Indeed, conventional techniques such as ultracentrifugation are time consuming to perform, inefficient for isolating EVs and not suitable for use on clinical samples.

The present inventors have extensively studied EVs as diagnostics/biomarkers of diseases such as breast cancer. The use of EVs to identify cancer-related upregulated proteins and diagnose a disease state is described in WO 2020/180896, which is incorporated by reference herein in its entirety for its teachings regarding the same. There, it was identified that EVs secreted from tumor cells into the bloodstream contained information/cargoes indicative of cancer. By detecting upregulated and/or elevated levels of cancer-related proteins in plasma EVs, such EV cargo can be used as a clinical breast cancer diagnostic.

The present disclosure applies a similar concept to ADME enzymes, which has not heretofore been possible for several reasons, some of which are provided above (inefficient techniques, low abundance of proteins due in part to other untargeted proteins and metabolites in biofluids, and results are often not reproducible). It is widely recognized that certain ADME enzymes are highly induced under specific conditions (e.g., liver disease, lung cancers, HIV infection, alcohol use, smoking and the use of other illicit drugs). For example, CYP2E1 is induced in chronic alcohol users and CYP1A1, CYP1B1 are induced/upregulated in smokers, and CYP3A4 is induced by several therapeutic drugs such as phenobarbitals, rifampin, and ritonavir. Similarly, biomarkers for ADME pathways can be used to characterize variability in drug exposure. However, conventionally, the source of ADME proteins for assessment have been from tissue (e.g., liver tissue) or microsome/cell lysate rather than biofluid. While human serum for example is a rich source of readily accessible EVs, the separation of EVs from serum proteins and non-EV lipid particles has been a considerable challenge using conventional techniques. Accordingly, tissue biopsies have been the standard and require invasive sample collection methods, which is not viable for clinical or point of care usage.

However, it has recently been established that EVs enriched with ADME enzymes circulate in biofluids. For example, Rowland et al., Plasma extracellular nanovesicle (exosome)-derived biomarkers for drug metabolism pathways: a novel approach to characterize variability in drug exposure, Br J Clin Pharmacol 85: 216-226 (2019) (the “Rowland Publication”), which is incorporated herein by reference in its entirety, reports that exosomes isolated from human plasma contained functional protein and mRNA for multiple CYP and UGT enzymes, including CYP1A2, 2B6, 2C8, 2C9, 2C19, 2D6, 2E1, 2J2, 3A4, and 3A5, UGT1A1, 1A3, 1A4, 1A6, 1A9, 2B4, 2B7, 2B10, and 2B15. The Rowland Publication also observed a strong concordance between induction of basal CYP3A4 expression in EV-derived exosomes and HepaRG cell lysate and administration of rifampicin, a well-known inducer (a 22- and 15-fold increase). Similarly, CYP2E1 and P450 protein expression levels in the exosome have been determined to equate with the administration of alcohol. (See, e.g., Cho et al., Increased ethanol-inducible cytochrome P450-2E1 and cytochrome P450 isoforms in exosomes of alcohol-exposed rodents and patients with alcoholism through oxidative and endoplasmic reticulum stress. Hepatology Communications 1(7): 675-690 (2017), which is incorporated by reference in its entirety. Accordingly, there are several known drug-metabolizing enzymes of significant clinical importance and inducement thereof (e.g., identified by profiling the upregulated expression of EV-derived CYP and UGT enzymes) correlates with administration of a drug (i.e. rifampicin) having a therapeutic effect on the subject.

Accordingly, CYP and UGT enzymes are useful biological markers for not only diagnosing the conditions mentioned herein, but also as an indicator of a subject's present metabolism phenotype as it relates to a specific drug. By way of a non-limiting example, the liver is one of the main organs of the body involved in drug metabolism. It has been determined that hepatocytes secrete EVs that are then circulated in the bloodstream and plasma, and such EVs carry ADME proteins as described above.

As it is established that certain ADME protein expression (phenotype) is directly associated with a subject's drug metabolism efficiency, the methods and unique biomarkers provided herein enable the ADME protein information within the biofluid EVs (e.g., hepatocyte-derived EVs in the peripheral blood) to be assessed to reveal the subject's present drug metabolism efficiency. This EV proteome analysis can then be leveraged to determine drug efficacy and dose ranging for pharmaceutical or dosing studies, or in point of care or otherwise clinical setting for profiling a subject's current drug response in an effort to facilitate safe and effective therapeutic treatment.

The methods hereof can be performed on any type of biofluid sample obtained from a subject. Where the underlying goal of the method is to assess the safety or efficacy of a candidate drug (e.g., in a pharmaceutical or regulatory trial), the subject can comprise an individual in a cohort. Alternatively, where the method comprises a method for profiling a subject's current drug response, the subject can be, for example, a human patient who is receiving, or is to receive, a drug.

In at least one embodiment, the biofluid sample comprises any biofluid comprising EVs. In an exemplary embodiment, the biofluid sample comprises blood, plasma, serum, urine, saliva or CSF. It will be appreciated that the particular biofluid sample can be selected based on the particular administration route of the drug. For example, and without limitation, where a drug is delivered to the brain, the sample can comprise CSF. Where a drug is delivered to the neck of a subject, the sample can comprise saliva.

### Proteome Analysis

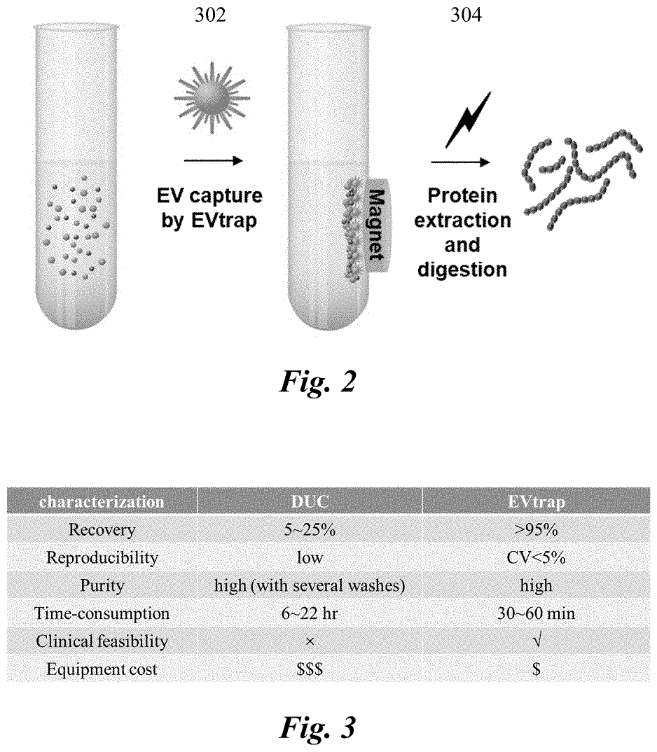

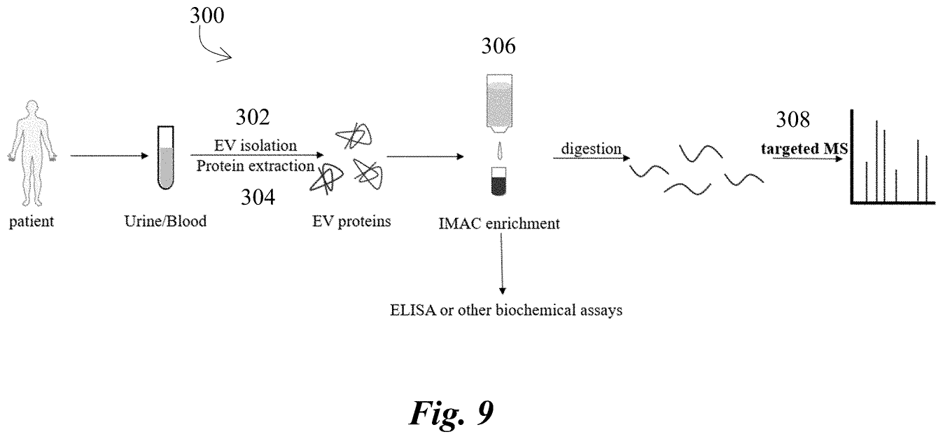

Now referring to FIGS. 2 and 9, a method 300 for proteome analysis comprises isolating EVs from a sample of biofluid and extracting (e.g. in situ) one or more intravesicle components (e.g., certain ADME proteins of interest) from the isolated EVs. Any EV isolation method now known or hereinafter developed can be used including, without limitation, differential ultracentrifugation (DUC), size-based isolation techniques such as ultrafiltration and high-performance liquid chromatography (HPLC), precipitation such as polymer-based precipitation, size exclusion chromatography (e.g., using qEV columns or iodixanol density gradient centrifugation) and/or filter-like membranes, affinity-based capture techniques, microfluidics-based isolation techniques and/or a combination of any of the foregoing.

In at least one exemplary embodiment, an EVTRAP platform can be employed as described in U.S. Patent Publication Number 2019/0301985, which is incorporated by reference herein in its entirety for its teachings regarding the same. FIG. 2 shows a diagram representative of one embodiment of such an EVTRAP platform, which utilizes beads with surfaces coated by combination of hydrophilic and lipophilic or hydrophobic groups to afford an affinity toward lipid-coated EVs.

When the sample is introduced into the EVTRAP, the EVs are captured (302) by the beads and isolated from the remainder of the sample (which can be washed away). This facilitates the washing step and the magnetic feature of the EV trap enables the captured EVs to be pulled to one side, facilitating quick separation of the EVs from the remainder of the sample.

The ADME proteins are then extracted from the trapped EVs (304) using standard extraction techniques including, for example, by a surfactant-based solution, triethylamine elution, on-beads digestion, high temperature elution, electric pulse-based extraction or similar techniques. Other extraction solutions can include urea, thiourea, Triton-X, sodium dodecyl sulfate (SDS), deoxycholate or any other molecules and conditions capable of disrupting the lipid membrane of the EVs or eluting intact EVs off the beads of the EVTRAP. The extracted/isolated EV cargo can include proteins, post-translationally modified proteins, DNA, mRNA, miRNA, metabolites, gene mutations and the like.

Now referring back to the proteome analysis method 300, after capture and extraction, the internal EV cargo (i.e. the ADME proteins) are enriched (step 306) and analyzed (step 308). Step 306 can be performed using affinity purification with specific antibodies, aptamers, or small molecules/nanopolymers, using metal-ion based methods (PoryMA, immobilized metal affinity chromatography (IMAC), TiC¾, ZrQz, A1203, SnO, etc.), organic extraction (e.g., TRIzol by Invitrogen Corporation, Carlsbad Calif.), antibodies (immunoprecipitation (IP) such as pTyr antibody IP), aptamers, and/or chromatography-based separation (HILIC, ERLIC, SCX, SAX, etc.).

Step 306 may be performed for example by affinity purification techniques. In at least one exemplary embodiment, step 306 is performed via IMAC, using at least three sequential washings. In other embodiments, step 306 is performed via IMAC and/or immunoaffinity chromatography.

In at least one embodiment, the enrichment step 306 is performed at least twice or three times in succession using various techniques. In certain embodiments, the steps of capture (302), extraction (304), and enrichment (306) are all considered sequential enrichments. This multi-step process can be especially advantageous depending on the type of biofluid sample analyzed and, in certain cases, can enhance the sensitivity of the method 300.

Eluted ADME proteins are analyzed (e.g., identified and quantified) at step 308 and identified using, for example, liquid chromatography-tandem mass spectrometry (LC-MS/MS), ELISA, microarrays, Western Blot, PCR RT-PCR, NGS, spectroscopy, NTA, DLS, binding probes, and/or other similar methods. In at least one embodiment, step 308 comprises analysis of the ADME proteins by LC-MS/MS on a high-speed and high-resolution mass spectrometer with technical replicates, followed by label-free quantification of the peptides to determine differential ADME protein expression.

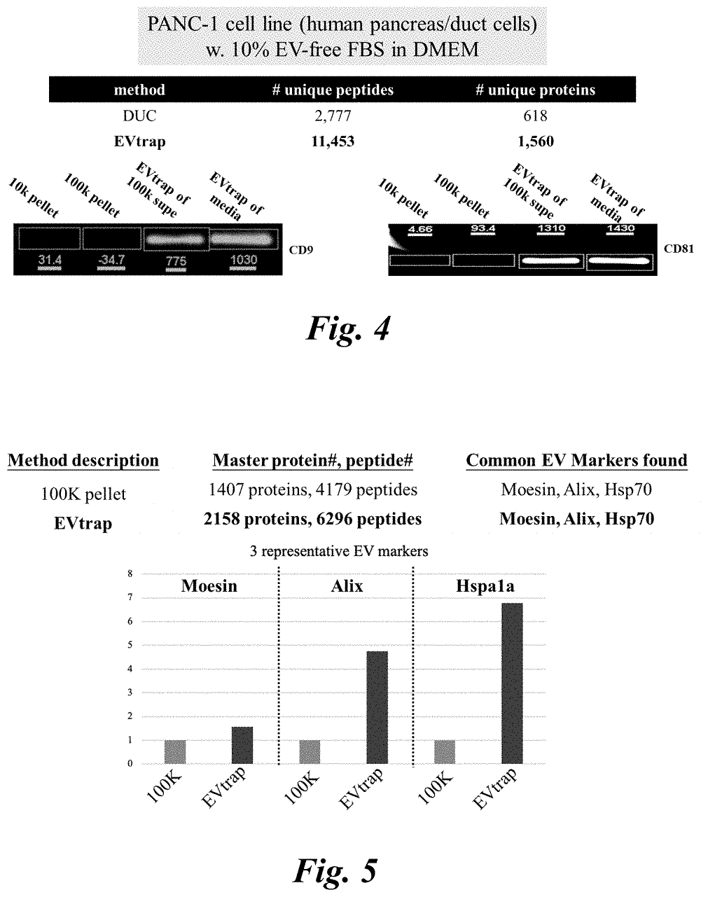

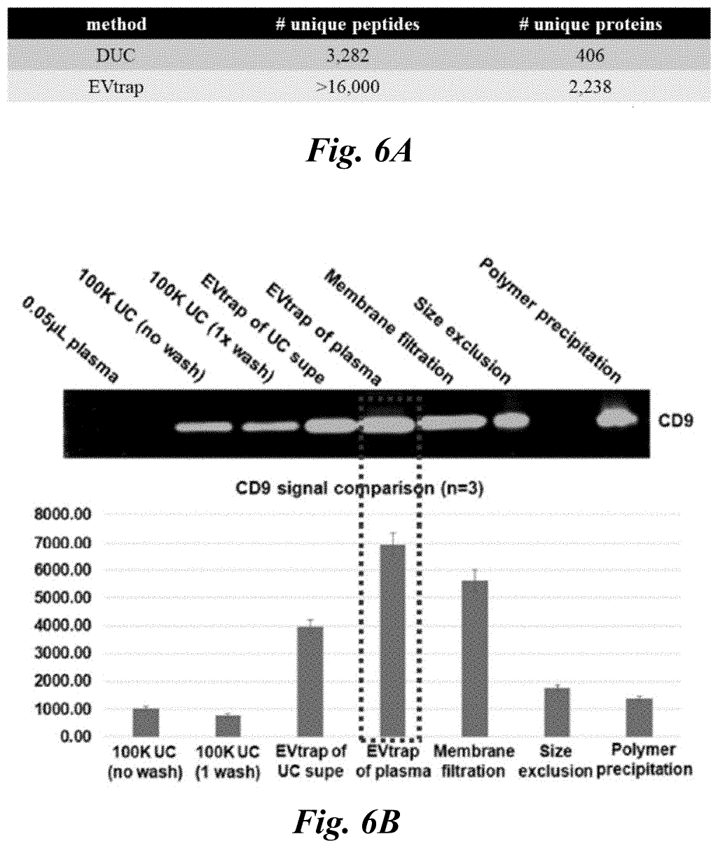

As supported by FIGS. 3-6D, the EVTRAP technique has overcome many of the challenges in EV isolation that were commonly seen with other conventional techniques. As shown in FIG. 4, the EVTRAP technique significantly outperformed DUC when both were used to isolate human pancreas/duct cell (PANC-1 cell line)-derived EVs suspended in 10% EV-free fetal bovine serum (FBS) and Dulbecco's Modified Eagle Medium (DMEM). There, common ADMEs such as CYP2A6, 2E1, 3A4, 3A5, 3A7, and 26C1 were identified in the hepatocyte media EVs in data-dependent acquisition (DDA) mode with a full-scan MS.

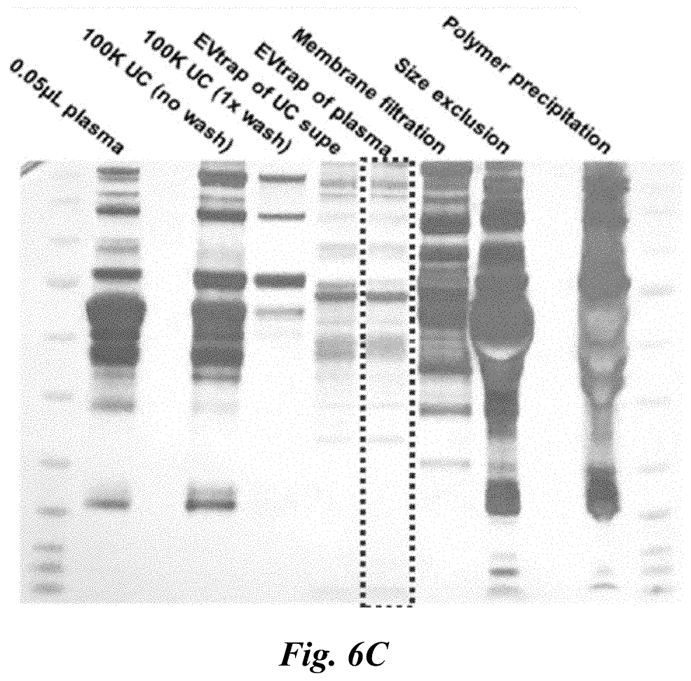

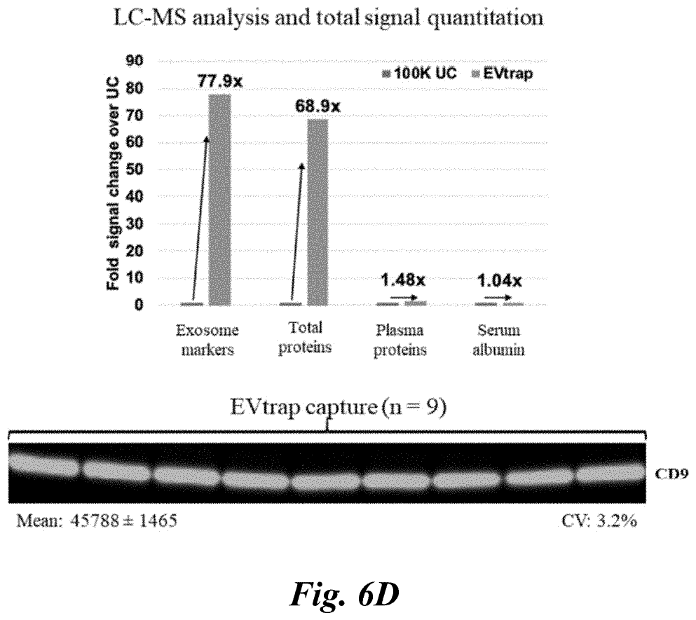

Similarly, the EVTRAP technique again significantly outperformed DUC when both were used to isolate mouse liver tissue-derived EVs (FIG. 5) and EVs from human plasma (FIG. 6A). FIGS. 6B-6D illustrate the high recovery of the EVTRAP technique (FIG. 6B), as well as the high purity of the results (FIGS. 6C and 6D). Indeed, as shown in FIG. 6D, the EV protein cargo abundance was significantly elevated when the EVTRAP technique was utilized, with contaminant free proteins remaining at the same level.

### Quantitative Analysis and Identification of Unique ADME Biomarkers

Having established a methodology for effectively measuring ADME protein cargo from biofluid samples, specific biomarkers that upregulate upon drug induction were then identified through quantitative data analysis. However, with respect to analysis, DDA mode is not applicable to identify and quantify ADME protein cargo in plasma EVs due to their overall low abundance/trace amount present in the samples. As opposed to conventional techniques that required get fractionation to increase assay sensitivity, a targeted quantitative mass spectrometry (MS) approach (parallel reaction monitoring (PRM) and affinity enrichment) was utilized, which proved much more sensitive. Generally, PRM is an ion monitoring technique based on high-resolution and high-precision MS and is useful for the absolute quantification of proteins and peptides, especially for the quantification of multiple proteins in a complex sample.

Primarily, CYP3A4 was employed as a prototype of ADME due to it being an established drug metabolizing enzyme. PRM allowed for low-abundant ADME protein detection in biofluid samples. A group of unique CYP3A4 peptides were identified, including SEQ ID NOS: 1-5 (see Table 1).

TABLE 1
Selected Targets from Study #1

| | | CYP3A4 | SEQ |
|---|---|---|---|
| Patient | CYP3A4 | unique peptides | ID |
| ID | identified | identified | NO: |
| A | √ | APPTYDTVLQMEYLD | 1 |
| | | MVVNETLR | |
| | | VWGFYDGQQPVLAIT | 2 |
| | | DPDMIK | |
| B | √ | APPTYDTVLQMEYLD | 1 |
| | | MVVNETLR | |
| | | VWGFYDGQQPVLAIT | 2 |
| | | DPDMIK | |
| C | √ | VWGFYDGQQPVLAIT | 2 |
| | | DPDMIK | |
| D | x | N/A | |
| E | √ | APPTYDTVLQMEYLD | 1 |
| | | MVVNETLR | |
| | | VWGFYDGQQPVLAIT | 2 |
| | | DPDMIK | |
| F | √ | APPTYDTVLQMEYLD | 1 |
| | | MVVNETLR | |
| | | VWGFYDGQQPVLAIT | 2 |
| | | DPDMIK | |
| G | √ | APPTYDTVLQMEYLD | 1 |
| | | MVVNETLR | |
| H | √ | APPTYDTVLQMEYLD | 1 |
| | | MVVNETLR | |
| | | VWGFYDGQQPVLAIT | 2 |
| | | DPDMIK | |
| I | √ | APPTYDTVLQMEYLD | 1 |
| | | MVVNETLR | |
| | | VWGFYDGQQPVLAIT | 2 |
| | | DPDMIK | |

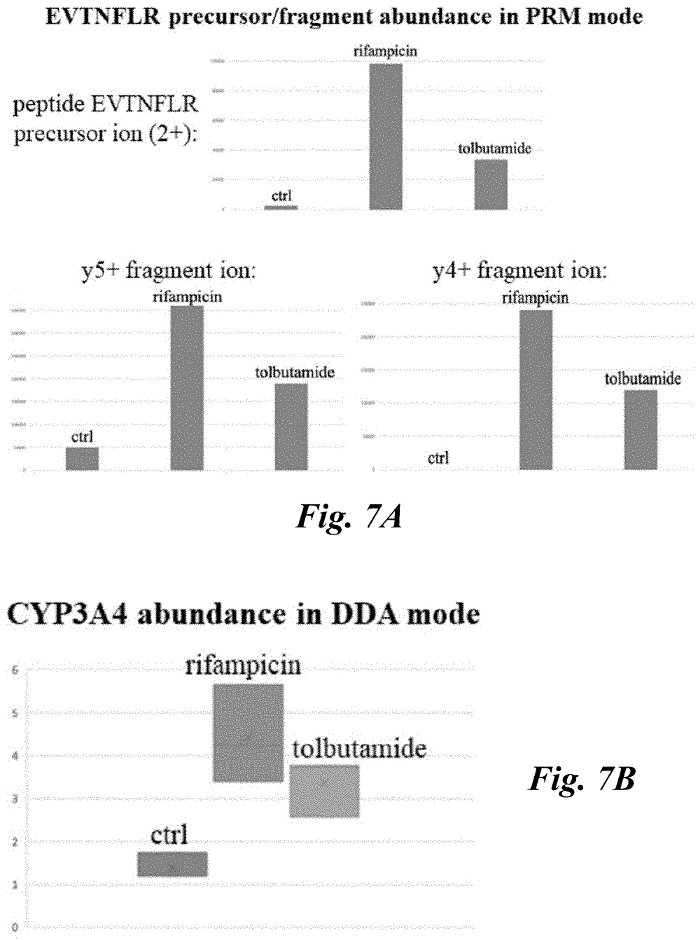

Using PRM, the identified biomarkers (EV-derived ADME proteins) were assessed following drug induction. Rifampicin was used as a well-known inducer and the biofluid samples were hepatocyte lysate. FIG. 7A indicates SEQ ID NO. 3 was upregulated in response to treatment with rifampicin. FIG. 7B reflects the same CYP3A4 data instead assessed in DDA mode, and clearly illustrates the significantly higher degree of sensitivity obtained through use of PRM in detecting EV-derived ADME changes in response to drug treatment.

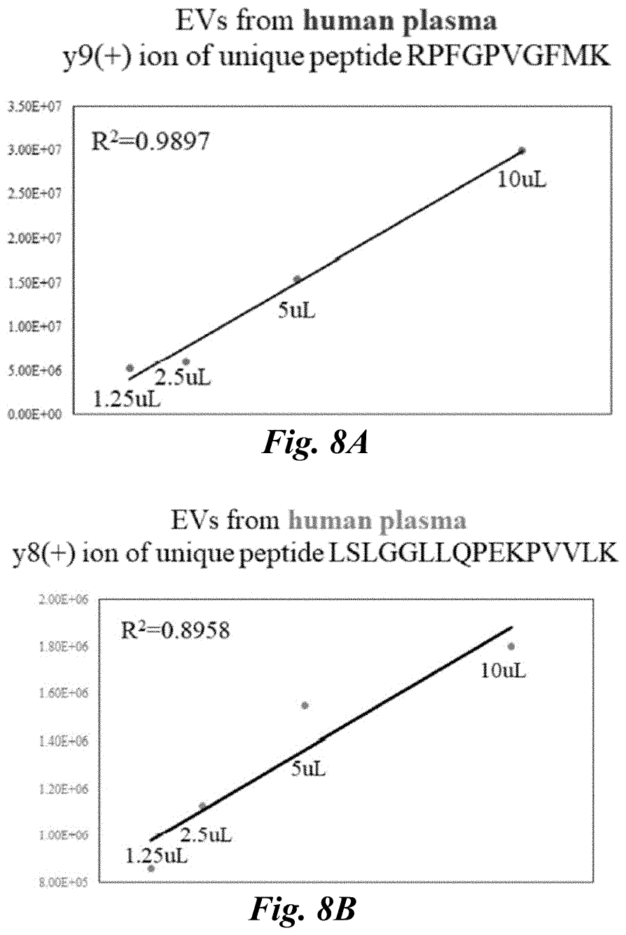

PRM produced similar results when applied to the detection and quantification of ADME proteins in plasma EVs. The EVs were isolated using the methods described in Example 1 below (specifically using the EVTRAP technique), with subsequent PRM analysis of the isolated ADME cargo. As shown in FIGS. 8A and 8B, CYP3A4 was quantified through PRM analysis on two specific peptides (SEQ ID NOS. 4 and 5). In both measurements, different amounts of human plasma were used to evaluate the quantitative measurement.

Accordingly, the targeted MS approach following EV isolation allows for the quantitative measurement of ADME proteins in biofluid EVs with sufficient sensitivity to enable clinical drug response detection. This can be leveraged not only in drug efficacy trials to determine dose ranging and efficacy of a compound, but also at point of care in a clinical setting to determine a patient's current drug response such that dosing and/or treatment regimen can be adjusted as needed.

In view of the above findings, methods are provided for determining drug efficacy and dose ranging of a compound (e.g., a drug). In at least one embodiment, a method for determining efficacy and dose ranging comprises selectively isolating a population of EVs in a biofluid sample taken from a subject having received at least one dose of a compound, extracting EV proteins from the isolated population of EVs, enriching the extracted EV proteins or peptides generated from the extracted EV proteins, and identifying and quantifying a level of expression of one or more targeted ADME proteins in the enriched EV proteins or peptides.

The targeted one or more ADME proteins or peptides can comprise CYP enzymes, UGT enzymes, acetyltransferase, an ATP-binding cassette transporter, a solute carrier transporter, or a combination of two or more of the foregoing. In at least one embodiment, the one or more targeted ADME proteins or peptides can comprise a protein or peptide having a sequence of one of SEQ ID NOS: 1-5.

The extracting step 304 of the method can be performed using any extraction technique described herein. In certain embodiments of the method, the extracting step 304 is performed using a surfactant-based solution, a triethylamine elution, on-beads digestion, a high temperature elution, or electronic pulse-based extraction. In at least one embodiment, the extraction step 304 further comprises generating EV peptides from the EV proteins. In such cases, the enriching and identifying/quantifying steps 306, 308 of the method 300 is performed on the extracted EV peptides rather than the EV proteins.

The enriching step 306 can likewise be performed using any enrichment techniques provided herein including, without limitation, enrichment by IMAC, antibodies (e.g., immunoaffinity chromatography), and/or IP. In at least one exemplary embodiment, the enriching step 306 comprises enriching the extracted EV proteins (or peptides, as appropriate) using affinity purification (e.g., IMAC or immunoaffinity chromatography).

In at least one exemplary embodiment, the isolating and extracting steps 302, 304 are performed using an EVTRAP as described herein and the enriching step 306 is performed by IMAC, antibody enrichment, and/or IP.

The identifying and quantifying steps (e.g., analysis step 308) can similarly be performed using any of the relevant techniques described herein. For example, and without limitation, the analysis step 308 can be performed by multi-reaction monitoring, parallel reaction monitoring or data-independent acquisition mass spectrometry. In at least one exemplary embodiment, the analysis step 308 is performed by parallel reaction monitoring (PRM).

As supported by the data set forth herein, upregulated expression of the targeted ADME protein(s) in the subject (as compared to a baseline expression of the targeted ADME protein(s); for example, established from a control or established from the subject prior to administration of the compound) correlates with the ADME protein(s) being active and, thus, the subject experiencing metabolic activity with respect to the administered compound. Accordingly, upregulated expression of the targeted ADME protein(s) is indicative of the at least one dose of the compound being within the therapeutic index of the compound for the subject (i.e. the subject experiencing a therapeutic response/the dose being a therapeutically effective dose).

Conversely, lack of upregulated expression of the targeted ADME protein(s) in the subject (i.e. the expression is baseline expression) is indicative of the at least one dose of the compound is outside of the therapeutic index of the compound. In other words, the targeted ADME protein(s) are not activated to a measurable extent and the compound dosage is not having a therapeutic effect on the subject.

Where lack of upregulated expression of the targeted ADME protein(s) is identified, in at least one embodiment, the method can further comprise adjusting a dosing range of the compound and/or timing of doses. For example, and without limitation, if the method indicates the at least one dose of the compound is not sufficient enough that it increases the activity of the targeted ADME protein(s) in the subject, the dose can be modified (e.g., increased or decreased in concentration and/or modified in timing and/or number of doses administered) in the interest of achieving metabolic activity of the compound in the subject and a therapeutic effect.

This method can be particularly useful in drug efficacy and safety studies as the present methods can provide timely and accurate details to (either alone or in conjunction with other clinical data) facilitate the adjustment of dose-response curves and in otherwise determining effective and safe dosing regimens for a particular compound. For example, where the method is applied to subjects that are members of a treatment cohort for a pharmaceutical dosing study, the data from all members of the treatment cohort can be assessed and the dose-response curves quickly repaired based on, for example, levels of the ADME proteins measured from such subject's blood or urine.

The present methods can further be used by healthcare providers at point of care to not only monitor a subject's response administration of a compound, but also to profile a subject's then-current (or present) drug metabolism phenotype. Accordingly, following the analysis step 308, the method 300 can further comprise assigning, based on the level of expression of the targeted one or more ADME proteins or peptides, the subject a phenotype for drug metabolism of, for example, poor, intermediate, extensive, or ultra-rapid. For example, and without limitation, where a low dose of a compound is administered to a subject (as determined, for example, based on known medical or regulatory data) and the subject exhibits upregulation (or activity) of the targeted ADME protein/peptide, a phenotype of extensive or ultra-rapid can be assigned to the subject and a dosing regimen of the compound can be designed and administered that takes such phenotype into account. Additionally, an alternative compound can be administered in lieu of the first compound (perhaps an alternative compound to which the subject is a more efficient or less sensitive metabolizer). Conversely, where a low dose of the compound is administered to the subject and the subject exhibits little or no activity/upregulation of the targeted ADME protein/peptide (as compared to baseline), a phenotype of intermediate can be assigned, and a dosing regimen based on such phenotype can be administered.

In at least one embodiment, where a subject exhibits little or no activity/upregulation of the targeted ADME protein/peptide (as compared to baseline) following administration of a first dose of a compound, the method can further comprise administering a second, adjusted dose of the compound to the subject (e.g., an increased dose concentration or dosage amount of the compound) and/or adjusting dose frequency to facilitate a therapeutic response in the subject. The steps of the method can be repeated as desired to monitor the effects of the adjusted dose on the subject and the dosage adjusted, as appropriate, depending on any identified upregulation or other activity (or inactivity) of the targeted ADME protein/peptide.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the present description, numerous specific details are set forth to provide a thorough understanding of the present disclosure. Particular examples may be implemented without some or all of these specific details and it is to be understood that this disclosure is not limited to particular biological systems, particular drugs, or particular biofluids, which can, of course, vary but remain applicable in view of the data provided herein.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Where a method of therapy comprises administering more than one treatment, compound, or composition to a subject, it will be understood that the order, timing, number, concentration, and volume of the administration is limited only by the medical requirements and limitations of the treatment (i.e. two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

### EXAMPLES

The following examples illustrate certain specific embodiments of the present disclosure and are not meant to limit the scope of the claimed invention in any way.

### Example 1: EV Isolation by EVTRAP (Step 302)

Urine collected from a human patient that had been administered a drug was processed pursuant to the workflow shown in FIG. 9 to identify upregulated CYP3A4. Upregulated CYP3A4 was used in these studies as a representative example of the method's use to extract and enrich targeted ADME proteins.

A defined volume of the wine sample was added to an EVTRAP loaded with buffer and incubated end-tip-end with EVTRAP magnetic beads at room temperature for 30 minutes. After supernatant removal using a magnetic separator rack, the beads were washed 2 times by washing with a first buffer, then 2 times by washing with a second buffer. Beads were rigorously vortexed in 100 μL 200 mM triethylamine for each elution to remove the underlying EVs, 2-time elution in total and combined. All elutes were fully dried in a vacuum centrifuge.

### Example 2: IMAC-Based Enrichment

Isolated EV samples were resuspended in the lysis solution (300 mM NaCl, 1% Nonidet P-40 (NP-40) in 50 mM Tris-HCL, pH˜8) and incubated 10 minutes at room temperature (step 304). At this point, (step 306) the samples were diluted for 20-fold by immobilized metal affinity chromatography (IMAC) loading buffer (50 mM Tris-HCL, 300 mM NaCl, pH˜8) and loaded onto Ni-NTA Spin Columns (Thermo Fisher Scientific, Wells Branch, Tex.) according to the manufacturer's instructions. After 3 sequential washes (loading buffer, 300 mM imidazole and 100 mM ethyl enediaminetetraacetic acid (EDTA)), the enriched proteins were eluted by NuPAGE LDS buffer (4×) at 95° C. and characterized by Western Blot (WB).

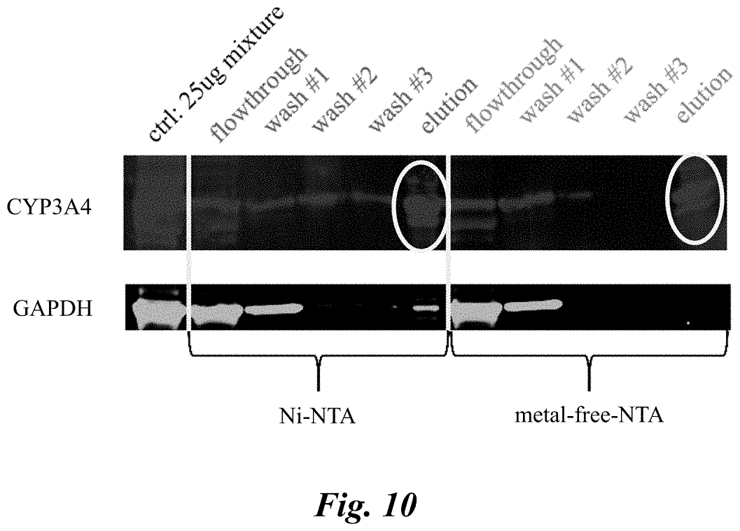

As shown in FIG. 10, the three-sequence enrichment was successful in substantially enriching the CYP3A4 within the EV samples, resulting in a detectable amount of CYP3A4. Further, as GAPDH was not selected for enrichment, the data supports that the enrichment was specific to CYP3 A4.

For MS analysis (step 308), enriched proteins were obtained through phase-transfer surfactant aided procedure Oncubation in elution of 12 mM sodium deoxycholate detergent (SDC) buffer, 12 mM sodium lauryl sulfate (SLS), 10 mM tris(2-carboxyethyl)phosphine (TCEP), and 40 mM chloroacetamide (CAA) in 50 mM Tris-HCl, pH 8.5 at 95° C. for 10 minutes).

### Example 3: Immunoprecipitation Enrichment

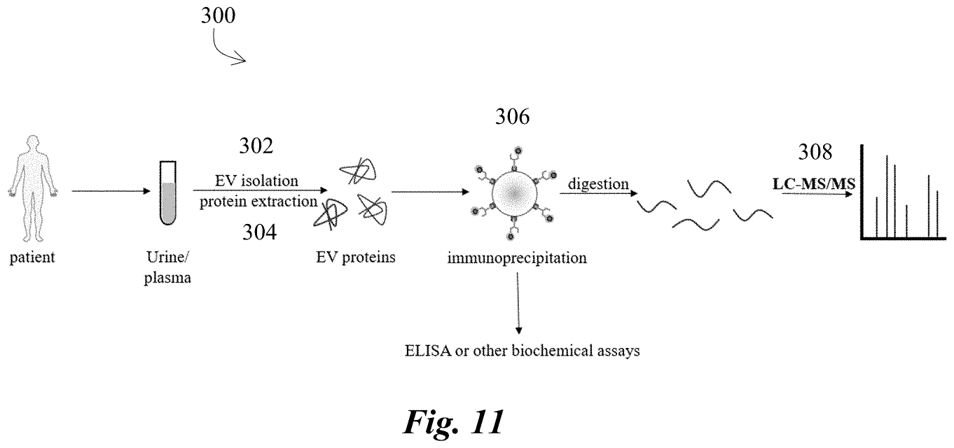

Urine collected from a human patient was processed to collect EVs pursuant to the workflow shown in FIG. 11 and as described in Example 1. At step 304, samples were solubilized in lysis buffer (12 mM SDC, 12 mM SLS in 50 mM Tris-HCL, pH 8.5) by vigorously shaking for 10 minutes at room temperature. After solubilization, the lysates were diluted 20-fold by IP lysis buffer (500 mM NaCl, 2 mM EDTA, 1×Thermo-protease inhibitor cocktail in 50 mM Tris-HCI, pH 7.5), washing using the same loading buffer, and incubated with Dynabeads® or protein A agarose beads (Thermo Fisher Scientific, Wells Branch, Tex.) according to the manufacturer's instructions. Via, capture from anti-CYP3A4 antibody (ab124921; Abcam, Cambridge, UK), purified proteins were eluted at 95° C. by LDS buffer and followed by WB detection. (Both Dynabeads® and agarose beads were immobilized with anti-CPY3A4 antibody.)

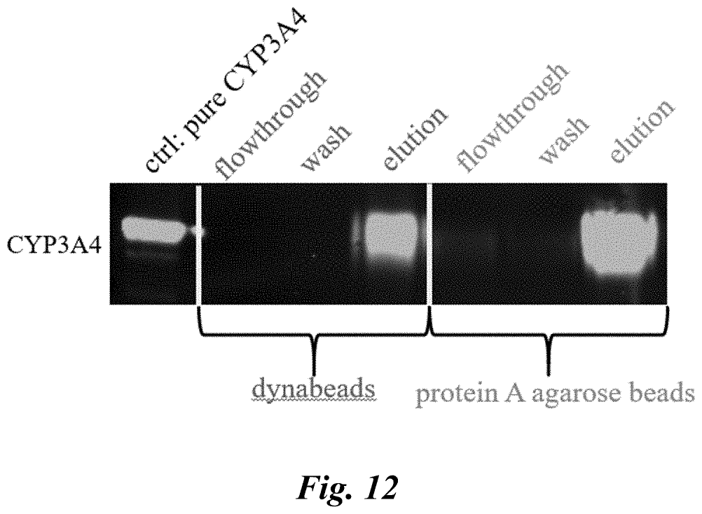

FIG. 12 confirms successful enrichment of the extracted CYP3A4 within the EV samples, resulting in a detectable amount of CYP3A4.

For MS analysis (step 308), purified proteins were collected by shaking in elution buffer (8 M Urea, 100 mM NaCl, 10 mM TCEP, and 40 mM CAA in 20 mM Tris-HCL, pH 7.5) at 37° C. for 45 minutes.

### Example 4: Western Blot Experiments

After boiling in LDS buffer, each sample was separated on an SDS-PAGE gel (NuPAGE 4-12% Bis-Tris Gel; Thermo Fisher Scientific, Wells Branch, Tex.). The proteins were transferred onto a low-fluorescence polyvinylidene difluoride (PVDF) membrane (MilliporeSigma, Burlingon, Mass.), and the membrane blocked with 1% bis)trimethylsilyl)acetamide (BSA) in Tris-buffered saline with polysorbate 20 (TBST).

A secondary antibody for visualizing the binding of the primary antibody was used, with Goat anti-Rabbit Alexa Fluor 800 nm (Thermo Fisher Scientific, Wells Branch, Tex), incubated for 1 hour in 1% BSA in TBST.

The membrane was scanned by an Odyssey near-infrared scanner (LI-COR Biosciences, Lincoln, Nebr.) for signal detection and quantitation.

### Example 5: LC-MS Sample Preparation

The extracted protein samples were diluted five-fold with 50 mM triethylammonium bicarbonate (TEAB) and digested with Lys-C (Wako Chemicals USA, Inc., Richmond, Va.) in a 1:100 (w/w) enzyme-to-protein ratio for 3 hours at 37° C., and sequentially in a final 1:50 (w/w) enzyme-to-protein ratio for overnight digestion at 37° C. Samples were acidified and trypsin deactivated with tritluoroacetic acid (TFA) to a final concentration of 1% TFA.

To remove detergents in the digested peptides (a step that was not necessary in urea elution conditions), ethyl acetate was added in a 1:1 ratio to the samples and the whole mixture vortexed for 2 minutes and then centrifuged at 20,000g for 2 minutes to obtain aqueous and organic phases.

The aqueous phase was collected and desalted, using Top-Tip C18 tips (GlyGen Corp, Columbia, Md.) according to the manufacturer's instructions.

1. A method for determining efficacy or dosing range of a compound comprising:
selectively isolating a population of extracellular vehicles (EVs) in a biofluid sample of a subject having received at least one dose of a compound;
extracting EV proteins from the isolated population of EVs;
enriching the extracted EV proteins or peptides generated from the extracted EV proteins; and
identifying and quantifying a level of expression of one or more targeted absorption, distribution, metabolism, and excretion (ADME) proteins in the enriched EV proteins or peptides;
wherein:
upregulated expression of the targeted one or more ADME proteins in the subject as compared to a baseline expression of the targeted one or more ADME proteins is indicative of metabolism by the subject of the compound and the at least one dose of the compound being within the therapeutic index of the compound for the subject, and
baseline expression of the targeted one or more ADME proteins in the subject is indicative of the at least one dose of the compound being outside of the therapeutic index of the compound for the subject.

2. The method of claim 1, further comprising adjusting a dosing range of the compound of the targeted one or more ADME proteins if the targeted one or more ADME proteins in the subject equates with baseline expression.

3. The method of claim 1, further comprising administering an adjusted dose of the compound to the subject if the targeted one or more ADME proteins has baseline expression in the subject.

4. The method of claim 1, wherein the step of extracting EV proteins further comprises generating EV peptides therefrom.

5. The method of claim 1, wherein the enriching step comprises enriching the extracted EV proteins or peptides generated from the extracted EV proteins using affinity purification.

6. The method of claim 5, wherein the affinity purification comprises immobilized metal affinity chromatography or immunoaffinity chromatography.

7. The method of claim 6, wherein the extracting step is performed via lipid affinity-based capture using a device comprising a plurality of microspheres having surfaces coated or modified with a combination of at least one hydrophilic group and at least one hydrophobic group.

8. The method of claim 1, wherein the biofluid sample is selected from a group consisting of urine, blood, cerebrospinal fluid, plasma, serum, tears, saliva, sweat, amniotic fluid, breast milk, ascites fluid, bile and cyst fluid.

9. The method of claim 5, wherein the biofluid sample comprises blood.

10. The method of claim 1, wherein the subject is an individual in a treatment cohort.

11. The method of claim 1, further comprising identifying the subject's phenotype for drug metabolism based on the quantified level of expression of the one or more targeted ADME proteins.

12. The method of claim 1, wherein the step of identifying and quantifying is performed by multi-reaction monitoring, parallel reaction monitoring, or data-independent acquisition mass spectrometry.

13. A method for profiling a subject's present drug response phenotype comprising:
selectively isolating a population of extracellular vehicles (EVs) in a biofluid sample of a subject that has received at least one dose of a first compound;
extracting EV proteins from the isolated population of EVs;
enriching the extracted EV proteins or peptides generated from the extracted EV proteins; and
identifying and quantifying a level of expression, using parallel reaction monitoring, of one or more targeted drug absorption, distribution, metabolism, and excretion (ADME) proteins in the enriched EV proteins or peptides; and
assigning the subject a phenotype for drug metabolism of poor, intermediate, extensive or ultra-rapid based on the level of expression of the targeted one or more ADME proteins.

14. The method of claim 13, further comprising the step of administering an adjusted dose of the first compound or administering at least one dose of a second compound based on the assigned phenotype.

15. The method of claim 13, wherein the step of extracting is performed using a surfactant-based solution, a triethylamine elution, on-beads digestion, a high temperature elution, or electronic pulse-based extraction.

16. The method of claim 13, wherein the targeted one or more ADME proteins comprise enzymes from the cytochrome P450 superfamily of hemoproteins (CYP), enzymes from the UDP-glucuronosyltransferse (UGT) family, or both CYP and UGT enzymes.

17. The method of claim 13, wherein the targeted one or more ADME proteins comprises CYP3A4.

18. The method of claim 13, wherein the targeted one or more ADME proteins comprise CYP enzymes, UGT enzymes, acetyltransferase, an ATP-binding cassette transporter, and a solute carrier transporter.

19. A method for determining drug efficacy comprising:
isolating a population of extracellular vehicles (EVs) in a biofluid sample of a subject having received at least one dose of a compound;
extracting EV proteins from the isolated population of EVs;
enriching the extracted EV proteins or peptides generated from the extracted EV proteins; and
identifying and quantifying a level of expression of one or more targeted drug absorption, distribution, metabolism, and excretion (ADME) proteins in the enriched EV proteins or peptides;
wherein:
upregulated expression of the targeted one or more ADME proteins in the subject as compared to a baseline expression of the targeted one or more ADME proteins is indicative of the at least one dose of the compound being within the therapeutic index of the compound for the subject, and
baseline expression of the targeted one or more ADME proteins in the subject is indicative of the at least one dose of the compound being outside of the therapeutic index for the subject; and
wherein the step of isolating is performed via lipid affinity-based capture using a device comprising a plurality of microspheres having surfaces coated or modified with a combination of at least one hydrophilic group and at least one hydrophobic group.

20. The method of claim 19, wherein the step of identifying and quantifying is performed by multi-reaction monitoring, parallel reaction monitoring, or data-independent acquisition mass spectrometry.
